(19) United States
(12) Patent Application Publication
(10) Pub. No.: US 2026/0284264 A1
(43) Pub. Date: Sep. 24, 2026

(54) METHOD FOR PRODUCING HUMAN ACELLULAR DERMAL MATRIX

(71) Applicant: CGBIO CO., LTD., Seoul (KR)

(72) Inventors: Hong Sun YOON, Yongin-si (KR); Jiwon WOO, Bucheon-si (KR); Kwon Young LEE, Seongnam-si (KR)

(21) Appl. No.: 19/469,911

(22) PCT Filed: Mar. 27, 2024
(86) PCT No.: PCT/KR2024/003881

§ 371 Date: Sep. 26, 2025

(30) Foreign Application Priority Data
Mar. 27, 2023 (KR) 10-2023-0039706
Mar. 5, 2024 (KR) 10-2024-0031253

(51) Int. Cl. A61L 27/36

(52) U.S. Cl. CPC A61L 27/3687; A61L 27/362

(57) ABSTRACT

The present invention relates to a method for producing a human acellular dermal matrix, wherein secondary washing is performed with an alcohol-based delipidation solution to provide excellent safety with respect to cytotoxicity and effectively remove residual fat. An object of the present disclosure is to provide a method for preparing the human body-derived acellular dermal matrix capable of effectively removing fat remaining in the dermis.

### CROSS-REFERENCE TO RELATED APPLICATIONS

This application is the National Stage filing under 35 U.S.C. 371 of International Application No. PCT/KR2024/003881, filed on Mar. 27, 2024, which claims the benefit of Korean Patent Application No. 10-2023-0039706, filed on Mar. 27, 2023, and No. 10-2024-0031253, filed on Mar. 5, 2024, the contents of which are all hereby incorporated by reference herein in their entirety.

### TECHNICAL FIELD

The present disclosure relates to a method for preparing a human body-derived acellular dermal matrix.

### BACKGROUND ART

Acellular dermal matrix (ADM) is for removing intradermal cells after removing the epidermis from donated cadaveric skin tissue in order to eliminate immune rejection, and it is widely used in soft tissue reconstruction in the relevant field, and is widely used also in an allograft for burn treatment. Dermal tissue is composed of 80 to 90% of collagen, elastin, and glycosaminoglycans.

Meanwhile, LifeCell developed a process for treating fragmented skin tissues with 1 and performing incubation at 37° C. for 18 to 32 hours for human skin and 35 to 55 hours for porcine skin to remove the epidermal tissues, decellularizing the tissues by treating them with a decellularization solution containing 0.5% SDS HBSS and 1 mM of EDTA at 40±5 rpm conditions for 1 hour, and treating the tissues with a cryopreservation solution. However, there have been problems in that applying NaCl to remove the epidermis necessitates a separate process solely for epidermal separation, which not only lengthens the process time but also hinders proper separation of the epidermis and dermis.

To solve the aforementioned problems, L&C Bio replaced NaCl with neutral proteolytic enzymes such as dispase, thermolysin, trypsin, etc., while Decell Technologies Inc. replaced NaCl with a hypotonic solution, thereby shortening the process time, but there have been problems in that failure to control the use concentration and treatment time resulted in tissue damage and residual fat within the dermis.

Therefore, there is a desperate need to develop a method that can remove residual fat without damaging the tissues.

### DISCLOSURE

### Technical Problem

An object of the present disclosure, which has been derived to solve the above-described problems, is to provide a method for preparing a human body-derived acellular dermal matrix capable of effectively removing fat remaining in the dermis.

### Technical Solution

In order to achieve the above object, a method for preparing a human body-derived acellular dermal matrix according to one embodiment of the present disclosure includes steps of (i) preparing a human body-derived skin tissue on which separate de-epidermization and delipidation processes have not been performed, (ii) immersion-treating the skin tissue in a hypotonic solution containing a surfactant, (iii) primarily washing the skin tissue that has been immersion-treated in the step (ii) with an isotonic solution, and (iv) secondly washing the skin tissue that has primarily been washed in the step (iii) with a delipidation solution.

### Advantageous Effects

According to one embodiment of the present disclosure, the method for preparing a human body-derived acellular dermal matrix not only can exhibit excellent safety for cytotoxicity, but also can effectively remove residual fat by performing secondary washing with an alcohol-based delipidation solution.

In addition, third washing is performed with purified water, thereby reducing the residual amount of IPA to a low toxicity level to enable safety to be improved.

In addition, tissue damage can be minimized, detergent residual amounts within the tissues can be reduced, and the safety for cytotoxicity can be excellent.

### BRIEF DESCRIPTION OF DRAWINGS

FIG. 1 is a process diagram for explaining a method for preparing a human body-derived acellular dermal matrix according to an embodiment of the present disclosure.

FIG. 2 is photographs showing the results before and after removing residual fat with IPA.

FIGS. 3A and 3B are photographs showing the results according to Experimental Example 1.

FIGS. 4 and 5 are graphs showing the results according to Experimental Example 2.

FIG. 6 is a graph showing the results according to 3-1 in Experimental Example 3.

FIG. 7 is photographs showing the results according to 3-2 in Experimental Example 3.

FIGS. 8, 9, 10A, 10B, and 11 are graphs showing the results according to Experimental Example 4.

FIGS. 12A, 12B, and 12C are photographs showing the results according to Experimental Example 5.

FIG. 13A is a table showing samples, and FIG. 13B is a table showing the results according to Experimental Example 6.

FIGS. 14A, 14B, and 14C are graphs showing the results according to Experimental Example 7.

FIG. 15 is a graph showing the results according to Experimental Example 8.

### MODE FOR INVENTION

Hereinafter, the present disclosure will be described in detail as embodiments thereof with reference to the attached drawings. However, the following embodiments are presented as illustrative examples of the present disclosure, if a detailed description of a technology or configuration well known to those skilled in the art is deemed to unnecessarily obscure the gist of the present disclosure, such detailed description may be omitted, and the present disclosure is not limited thereby. The present disclosure is capable of various modifications and applications within the scope of the description of the claims described later and their equivalents interpreted therefrom.

In addition, the terminologies used in this specification are terminologies used to appropriately express preferred embodiments of the present disclosure and may vary depending on the intent of the user or operator, the practices, etc. of the field to which the present disclosure pertains. Therefore, definitions of these terminologies should be based on the content throughout this specification. Throughout the specification, when a part is referred to as “including” a component, this means that the part does not exclude other components, but rather includes other components, unless otherwise specifically stated.

Throughout this specification, the term “%” used to indicate the concentration of a specific substance, unless otherwise specified, refers to % (w/w) for solid/solid, % (w/v) for solid/liquid, and % (v/v) for liquid/liquid.

In the human body-derived acellular dermal matrix of the present disclosure, the dermal matrix may be bone, ligament, tendon, or skin, and may include a xenogeneic or allogeneic origin-derived dermal tissue.

Hereinafter, a method for preparing a human body-derived acellular dermal matrix according to one embodiment of the present disclosure will be described in detail along the drawings.

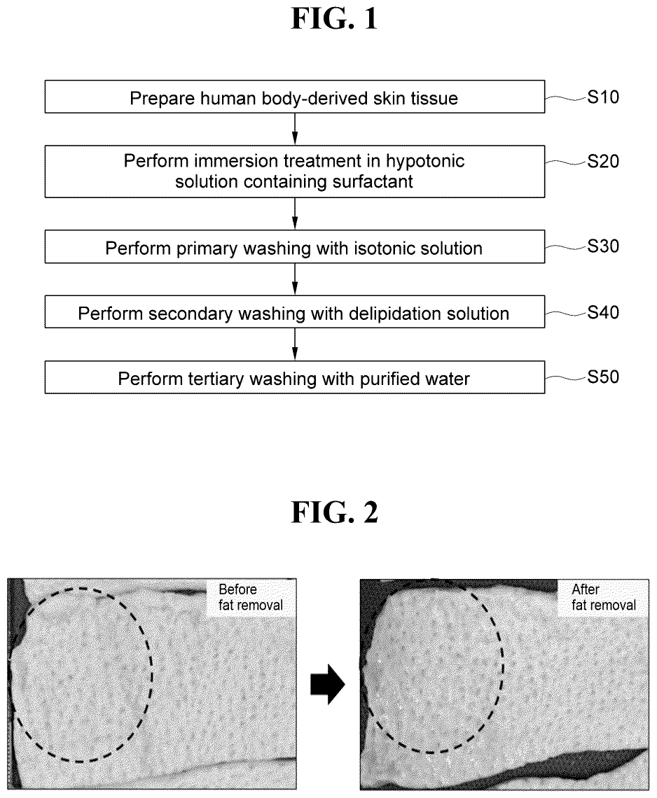

FIG. 1 is a process diagram for explaining a method for preparing a human body-derived acellular dermal matrix according to one embodiment of the present disclosure.

Referring to FIG. 1, first, a human body-derived skin tissue on which separate de-epidermization and delipidation processes have not been performed is prepared (S10).

Next, the skin tissue is immersion-treated in a hypotonic solution containing a surfactant (S20).

The skin tissue prepared in the step S10 may be immersion-treated in the hypotonic solution containing a surfactant for 2 to 24 hours, for example, 2 to 10 hours, another example, 3 to 9 hours, or another example, 4 to 8 hours.

In this embodiment, the “hypotonic” refers to a state in which the inside of a semipermeable membrane has a higher osmotic pressure than the outside, when comparing the osmotic pressures of two solutions with the membrane being interposed between the two solutions, i.e., a state in which net movement of water faces toward the inside of the membrane due to a state in which the concentration inside the membrane is higher than the concentration outside, and the rate of an osmosis phenomenon that is a passive diffusion phenomenon of water, increases or decreases in proportion to the concentration gradient between the two solutions, and if the concentration difference disappears, net movement of water may disappear.

In this embodiment, the “hypotonic solution” is used in a treatment for affecting cell lysis and may refer to a solution with an osmolarity of approximately 270 mOsm/L or less. For example, the hypotonic solution permeates the cell membrane through osmosis and flows into the cell, and at this time, the cells of the tissue may be caused to swell and rupture, thereby eliminating residual cells.

In this embodiment, the “osmolarity” refers to an expression of osmotic concentration per volume of solution (mOsm/L), and the osmotic concentration of plasma in the body and other body fluids may be 270 to 300 mOsm/L. If the osmolarity of a solution is about 270 mOsm/L or less, it may be defined as a hypotonic solution, if it is in a range of about 270 to 300 mOsm/L, it may be defined as an isotonic solution, and if it is about 300 mOsm/L or more, it may be defined as a hypertonic solution.

The hypotonic solution of this embodiment has an osmolarity of 270 mOsm/L or less, may preferably contain 5 to 20 mM of Tris-HCl, 0.1 to 1.5% of ethylenediaminetetraacetic acid (EDTA), and 0.01 to 0.05 M of sodium hydroxide (NaOH), and may more preferably contain 8 to 12 mM of Tris-HCl, 0.7 to 1.2% (w/v) of ethylenediaminetetraacetic acid (EDTA), and 0.01 to 0.02 M of sodium hydroxide (NaOH).

Meanwhile, the surfactant may include an ionic surfactant, a non-ionic surfactant, or a mixture thereof, and preferably may include an ionic surfactant. For example, ionic surfactants may include one or more selected from the group consisting of glutamate-based surfactants such as alkyl benzene sulfonate, alkyl sulfate, alkyl ether sulfate, alkoxylated amide, olefin sulfonate, alkyl xylene sulfonate, dialkyl sulfosuccinate, fatty acid ester sulfonate, alcohol sulfate, glycerol fatty acid ester, etc., isethionate-based surfactants, phosphate-based surfactants such as lauryl phosphate, laureth-1-phosphate, etc., taurate-based surfactants, alkoxylated alcohol-based surfactants, alkyl carboxylate-based surfactants, hydroxyalkyl, quaternary ammonium salt, and ethoxylated alkyl, preferably may include one or more selected from the group consisting of sodium dodecyl sulfate (SDS), sodium laureth sulfate (SLS), sodium lauryl ether sulfate (SLES), sodium myreth sulfate (SMES), dioctyl sodium sulfosuccinate (DSS), perfluorooctanesulfonate (PFOS), perfluorobutanesulfonate (PFBS), perfluorononanoate (PFNA), perfluorooctanoate (PFOA), sodium stearate, sodium lauroyl sarcosinate, cetrimonium bromide (CTAB), cetylpyridinium chloride (CPC), benzalkonium chloride (BAC), benzethonium chloride (BZT), dimethyldioctadecylammonium chloride (DDAC), and dioctadecyldimethylammonium bromide (DODAB), and more preferably may be sodium dodecyl sulfate (SDS), but the present disclosure is not limited thereto.

Conventionally, cellular components other than proteins from tissues have been removed by a method of dissolving cell membranes using non-ionic surfactants or high-concentration ionic surfactants, but there have been problems in that denaturation of residual proteins within the scaffold is caused, the extracellular matrix microstructure is collapsed, and growth factors are excessively removed. Therefore, in this embodiment, the aforementioned problems were solved by applying low-concentration ionic surfactants. Specifically, a low-concentration alkyl sulfate, more preferably a low-concentration sodium dodecyl sulfate (SDS), was used as the ionic surfactant. The concentration of the sodium dodecyl sulfate (SDS) in the hypotonic solution may be 0.1 to 0.5% (w/v), preferably 0.2 to 0.4% (w/v), but the present disclosure is not limited thereto.

The skin tissue is then primarily washed with an isotonic solution (S30).

In the step S20, the immersion-treated skin tissue can be primarily washed with an isotonic solution 1 to 10 times for 1 to 24 hours each, for example, 1 to 10 times for 1 to 10 hours each, and for another example, 2 to 7 times for 2 to 7 hours each.

In this embodiment, the isotonic solution has an osmolarity of about 270 to 300 mOsm/L, preferably may contain 25 to 100 mM of Tris-HCl, 0.1 to 1.5% (w/v) of ethylenediaminetetraacetic acid (EDTA), 0.01 to 0.05 M of sodium hydroxide (NaOH), and 0.05 to 0.3 M of sodium chloride (NaCl), and more preferably may contain 45 to 55 mM of Tris-HCl, 0.8 to 1.2% (w/v) of ethylenediaminetetraacetic acid (EDTA), 0.01 to 0.02 M of sodium hydroxide (NaOH), and 0.05 to 0.3 M of sodium chloride (NaCl).

NaCl in the above-described isotonic solution may be applied to a concentration of 0.05 to 0.5 M, preferably 0.1 to 0.3 M, but if the isotonic solution is deviated from the above-described range, the epidermis may not separate properly depending on the skin's raw materials.

In this embodiment, the “isotonic” refers to a state in which the osmotic pressures of the solutions with a semipermeable membrane being interposed between the solutions are the same, i.e., a state in which the concentration is the same so that there is no net movement of water. If the concentrations of the solutions with the membrane being interposed between the solutions are different, water will move from a place of a lower concentration to a place of a higher concentration, and the rate of the osmosis phenomenon, which is such a passive diffusion phenomenon of water, may be proportional to the concentration gradient between the two solutions.

A step of performing washing using PBS between S20 and S30 and between S30 and S40 for 6 to 24 hours, or performing washing 1 to 10 times for 5 to 60 minutes each may be further included.

Thereafter, the primarily washed skin tissue is secondarily washed with a delipidation solution (S40).

The skin tissue primarily washed in the step S30 can be secondarily washed with a delipidation solution having a concentration of 50 to 100%, preferably 80 to 100%, at 4 to 40° C., for example, 20 to 40° C., and for another example, 30 to 40° C., for 2 to 24 hours, for another example, 3 to 24 hours, and for another example, 3 to 5 hours. If the concentration of the delipidation solution is less than 50%, the fat removal rate may be as low as 20% or less, which is not preferred. If the washing time is less than 2 hours, the fat removal rate may be as low as less than 20%, and if it exceeds 24 hours, the fat removal rate may not be that great compared to the time increase rate. If the washing temperature is less than 4° C., the fat removal rate may be very low, 10% or less, and if it exceeds 40° C., the skin tissue may be denatured.

When the preferred concentration of the delipidation solution and the preferred temperature and time conditions for the secondary washing are all satisfied, the fat removal rate can be very excellent to be 58% or higher.

For example, the delipidation solution may include a polar solvent, wherein the polar solvent is an alcohol-based solvent, and may include one or more selected from preferably isopropyl alcohol (IPA), ethanol, methanol, butanol, octanol, and water, and preferably isopropyl alcohol.

Finally, the secondary washed skin tissue is tertiarily washed with purified water (S50).

The skin tissue secondarily washed in the step S40 can be tertiarily washed with purified water for 4 to 24 hours, preferably 6 to 24 hours. If the washing time is less than 4 hours, the remaining delipidation solution in the skin tissue may exceed 8,000 ppm to have toxicity, and if the washing time exceeds 24 hours, the decrease in the residual rate of the delipidation solution may not be that great compared to the time increase rate. If the above-described preferred time is satisfied, the residual rate of the delipidation solution may be in a safe state at 1,500 ppm or less.

In this embodiment, purified water may be sterilized distilled water (DW).

Hereinafter, the present disclosure will be described in more detail using examples. These Examples are intended solely to illustrate the present disclosure more specifically, and it will be apparent to those skilled in the art that the scope of the present disclosure is not limited by these Examples.

### Example 1. Preparation of Acellular Dermal Matrix

Skin tissues were purchased from the EURO skin bank, Allosource, and CTS to select tissues with a thickness of 1 mm or more, adipose tissues attached to skin tissues were removed using forceps, and then they were washed three times with aseptic water. The tissues were then immersion-treated in a hypotonic solution (10 mM of Tris-HCl, 1 g of EDTA, 13.5 mL of 1 M NaOH) containing 0.25% SDS for 6 hours. The skin tissues were then washed with PBS at 4° C. to remove fat, epidermis, cells, and hypotonic solution remaining on the skin tissues, and after leaving them overnight, the tissues were primarily washed with an isotonic solution (50 mM of Tris-HCl, 1 g of EDTA, 0.15 M of NaCl, 13.4 mL of NaOH) for 6 hours to prepare a human body-derived acellular dermal matrix.

### Examples 2 to 5. Preparation of Acellular Dermal Matrix with Residual Fat Removed

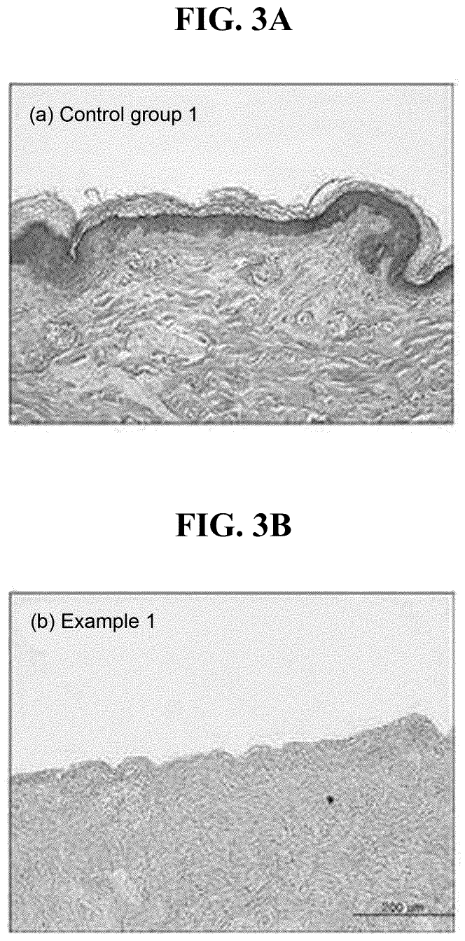

Secondary washing was performed on the human body-derived acellular dermal matrix prepared in Example 1 under the conditions described in Table 1 below, and tertiary washing was performed thereon with sterile purified water for 2 hours or more to prepare an acellular dermal matrix with residual fat removed, and before and after photos are as shown in FIG. 2.

TABLE 1

| | | | Secondary | |
|---|---|---|---|---|
| | IPA | Secondary | washing | |
| | concentration | washing | temperature | Tissue |
| Classification | (%) | time | (° C.) | formulation |
| Example 2 | 50 | 2 h | 20 to 25 | Slice |
| | | | | (1 to 2 mm) |
| Example 3 | 70 | 2 h | 20 to 25 | Slice |
| | | | | (1 to 2 mm) |
| Example 4 | 100 | 2 h | 20 to 25 | Slice |
| | | | | (1 to 2 mm) |
| Example 5 | 100 | 4 h | 4 | Whole |
| | | | | (2 to 3 mm) |
| Example 6 | 100 | 4 h | 20 to 25 | Whole) |
| | | | | (2 to 3 mm |
| Example 7 | 100 | 4 h | 38 | Whole |
| | | | | (2 to 3 mm) |
| Example 8 | 100 | 2 h | 38 | Slice |
| | | | | (1 to 2 mm) |
| Example 9 | 100 | 4 h | 38 | Slice |
| | | | | (1 to 2 mm) |
| Example 10 | 100 | O/N | 38 | Slice |
| | | | | (1 to 2 mm) |
| Example 11 | 100 | 4 h | 38 | Whole |
| | | | | (3 to 4 mm) |
| Example 12 | 100 | O/N | 38 | Whole |
| | | | | (3 to 4 mm) |
| Example 13 | 100 | 4 h | 38 | powder |

### Example 14. Preparation of a Composition Comprising a Fibrous Acellular Dermal Matrix

The acellular dermal matrix from Example 7 was tertiarily washed with sterile purified water for 4 hours instead of 2 hours, thereby preparing an IPA-removed acellular dermal matrix.

### Example 15. Preparation of an IPA-Removed Acellular Dermal Matrix

The acellular dermal matrix from Example 7 was tertiarily washed with sterile purified water for overnight instead of 2 hours, thereby preparing an IPA-removed acellular dermal matrix.

### Experimental Example 1. Decellularization Evaluation (H&E Staining)

Decellularization evaluation was performed on the human body-derived dermal matrix prepared in Example 1 through histological examination using H&E staining.

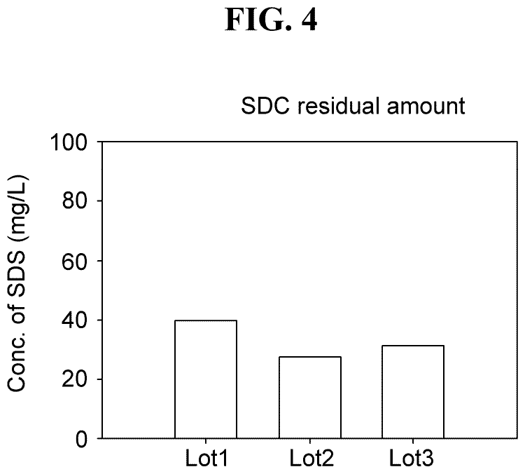

After a paraffin block was sectioned to 4 μm thickness, the sectioned paraffin block was dried to fabricate a paraffin section. Afterwards, for the deparaffinization process, it was reacted in xylene 3 times for 5 minutes each, 100% ethanol 3 times for 2 minutes each, 90% ethanol once for 1 minute each, 80% ethanol once for 1 minute each, and 70% ethanol once for 1 minute each, and then washed in running water for 10 minutes. After reacting the washed paraffin section in a hematoxylin staining solution for 10 minutes, it was washed in running water for 3 minutes, and after reacting it in an eosin staining solution for 10 minutes, it was washed in running water until no eosin staining solution came out. After it was reacted in 70% ethanol 10 times for 1 second each, 80% ethanol 10 times for 1 second each, 90% ethanol 10 times for 1 second each, 100% ethanol 2 times for 1 minute each, and xylene 3 times for 3 minutes each, and after the sample was mounted with a mounting solution, it was photographed with an optical microscope (Olympus BX51, H&E staining) and a scanning electron microscope (Hitachi S-4700, Japan), and the results are shown in FIGS. 3A and 3B.

Referring to FIGS. 3A and 3B, it could be confirmed in the case of Example 1 that the epidermis was peeled off compared to Control Group 1 (Fresh skin). In addition, as the results of comparing the decellularization effects of a hypotonic solution containing 1% Triton X-100, 1% Tween 80, 0.1% SDS solution, and 0.25% SDS under the same treatment time conditions, it could be confirmed that when treated with 1% Triton X-100, 1% Tween 80, and 0.1% SDS solution, cells of the skin tissue were not removed, and when treated with a hypotonic solution containing 0.25% SDS, cells within the tissue were mostly removed (data not shown).

In addition, it could be confirmed that in the skin tissue sonicated for 1 hour and the groups treated with ultrasonication and 1% Triton X-100 for 1 hour, cells were not removed and the tissue was damaged (data not shown). Accordingly, when skin tissue that had not undergone epidermal removal (using NaCl or proteolytic enzymes) and fat removal (using HIPS) processes was treated with surfactant solutions such as 1% Triton X-100, 1% Tween 80, and 0.1% SDS solutions, it could be seen that decellularization did not occur, and when treated with a hypotonic solution containing 0.25% SDS under the same conditions, it was confirmed that the epidermis, fat, and cells were removed in one step.

In other words, even when a physical process combining sonication and surfactants was performed to remove the epidermis, fat, and cells at once, it could be seen that the cells were not removed.

### Experimental Example 2: Detergent Residual Amount Assessment

A methylene blue dye binding assay was performed to assess the detergent residual amount in the tissue.

After drying the dermal tissue from the conventional process (samples in which the epidermis was removed using NaCl, fat was removed, and cells were removed using SDS) and Example 1, the detergent (SDS) residual amount was assessed using the eluates eluted with PBS.

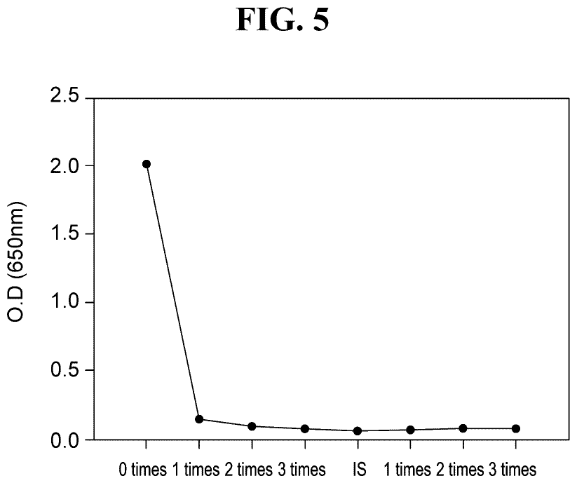

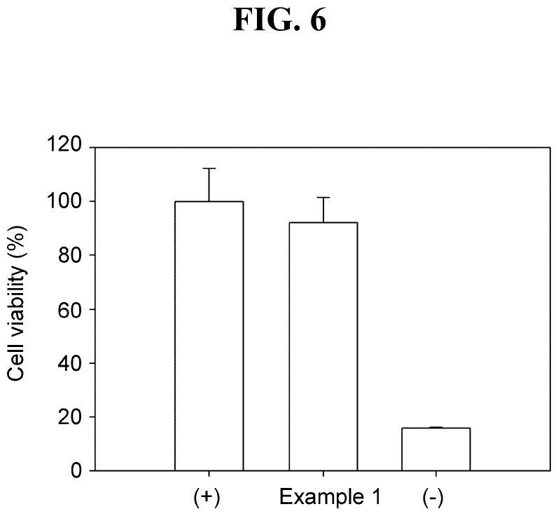

After the detergent (SDS) solution was diluted into methylene blue by concentrations (18, 0.58, 0.25%, 0.125%, 0.0625%, 0%), eluted with chloroform, and the absorbances at 650 nm were measured to create a standard curve, the 650 nm absorbances of the eluates eluted from Example 1 corresponding to the conventional and new processes were measured to check the detergent residual amounts, and the results are shown in FIG. 4 and Table 2. In addition, the residual amounts of the detergent were measured by taking the washing solutions from the dermal tissue from Example 1 at each washing step, and the results are shown in FIG. 5.

During the elution process, a dermal tissue having a thickness of approximately 3 mm and a weight of 4 g was selected, and 20 mL of an eluate was obtained at 37° C. for 72 hours.

Referring to FIG. 4 and Table 2, it could be confirmed that the residual detergent in the eluate from Example 1 was approximately 32.87±6.42 mg/L, which was lower than that of the conventional process (data not shown).

That is, it could be confirmed that the human body-derived acellular dermal matrix prepared according to the present disclosure exhibited an SDS detergent residual amount of 40 mg/L or less, as measured by absorbance at 650 nm for the eluate eluted with PBS.

TABLE 2

| Classification | New process (72-hour elution) | | |
|---|---|---|---|
| Residual | 39.9566 | 27.4441 | 31.1979 |
| amount (mg/L) | | | |

Referring to FIG. 5, it was confirmed that the amount of detergent in the washing solution decreased with each PBS wash.

### Experimental Example 3: Safety Evaluation (Cytotoxicity Analysis)

To evaluate safety through cell compatibility and toxicity, the MTT assay, and the MEM Elution Test in ISO10993-5: Cytotoxicity Test were performed.

### 3-1. MTT Assay (Cell Compatibility)

100 μL of L929 cells per well was dispensed in a 96-well plate at a concentration of 1×10⁵ cells/mL, and then, after 24 hours of culture, the eluate eluted from the existing process and the new process, Example 1, was treated with MEM culture medium and cultured for 24 hours at 37° C. 20 mL of an eluate was obtained by selecting a dermal tissue having a thickness of approximately 3 mm and a weight of 4 g in the existing process and the new process, and then eluting the eluate as a medium for animal cell culture at 37° C. for 24 hours.

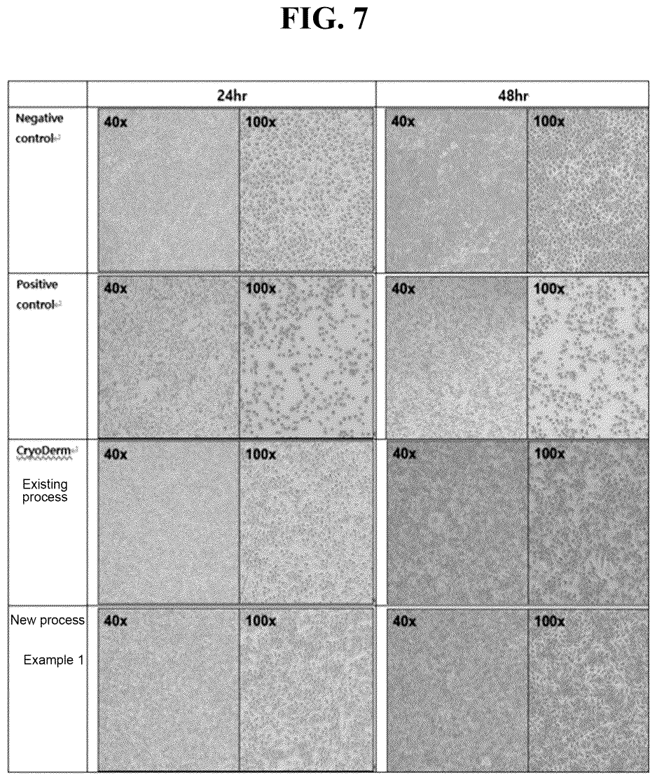

After 24 hours of culture of the eluate treatment, 5 mg/mL of 3-(4,5-dimethylthiazol-2-yl)-2.5-diphenyl tetrazolium bromide (MTT) reagent was diluted 1:100 with the culture medium, and 0.1 mL of this solution was added to each well and cultured for 5 hours. After 5 hours, the culture medium from each well of the 96-well plate was removed, and 0.1 mL of dimethylsulfoxide (DMSO) was added to each well to lyse the cells. Thereafter, the 96-well plate was placed in a spectrophotometer, and the absorbance was measured at a wavelength of 540 nm, and the results are shown in FIG. 6.

Referring to FIG. 6, the novel process, Example 1, showed cell viability values of 96.26%, 101.49%, and 94.99%. These are higher survival rate values compared to the existing process, and it can be seen that Example 1 has higher cell compatibility than the existing process.

That is, it could be confirmed that the human body-derived acellular dermal matrix prepared according to the present disclosure exhibited a very high cell viability of 90% or more, as measured by absorbance at 540 nm for the eluate eluted with MEM culture medium.

### 3-2. Minimum Essential Medium (MEM) Elution Test (Toxicity)

4 mL of L929 cells per well was dispensed in a 96-well plate at a concentration of 1×10⁵ cells/mL, and then, after 24 hours of culture, the monolayer culture status of the cells was checked. The cells were then treated with the eluates from the existing process and the novel process, Example 1, and cultured for 48 hours. As the eluates, a 20 mL eluate obtained by selecting a dermal tissue having a thickness of approximately 3 mm and a weight of 4 g from dermal tissues of the existing process and the new process, Example 1, and eluting it with an MEM culture medium at 37° C. for 24 hours was used. After 24 and 48 hours of culture, the positive and negative control materials were replaced with DMSO and MEM, respectively.

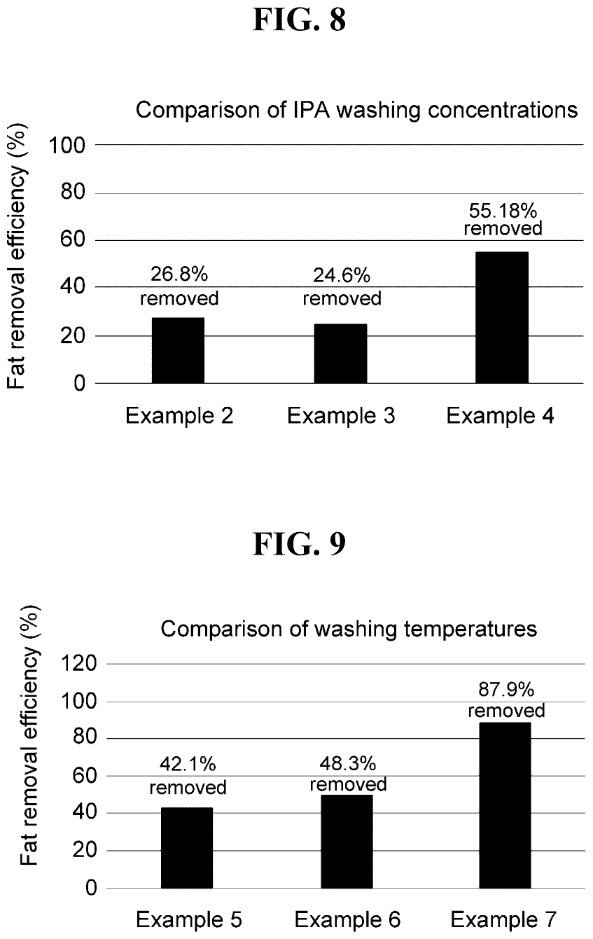

After 24 and 48 hours of culture, cell growth and lysis degrees were observed using a microscope, cell toxicity was graded according to Table 3 below, and the results are presented in FIG. 7 and Table 4. If the determination grade was 2 or lower, the test substance eluate was determined to have no cytotoxicity.

TABLE 3

| Grade | Reactivity | Condition of cultured cells |
|---|---|---|
| 0 | None | No separation of intracytoplasmic granules, |
| | | no cell lysis, no inhibition of cell growth |
| 1 | Very | The shape of cells is round, the cells are loosely |
| | weak | attached, intracytoplasmic granules are lost, or |
| | | cells showing morphological changes do not exceed |
| | | 20%. Occasionally, lysed cells are present and |
| | | slight growth inhibition is observed. |
| 2 | Weak | The shape of the cells is rounded, cells in which the |
| | | intracytoplasmic granules are lost do not exceed |
| | | 50%, and there is no extensive cell lysis. The |
| | | growth inhibition of the cells does not exceed 50%. |
| 3 | Severity | The shape of the cells is rounded, or lysed cells |
| | | do not exceed 70%. Cell layers are not completely |
| | | destroyed, but 50% or more of cell growth inhibition |
| | | is shown. |
| 4 | Extremely | The cell layers are almost or completely destroyed |
| | severe | |

Referring to FIG. 7 and Table 4, although Example 1 was rated as Grade 0 even after 48 hours, it could be confirmed that the existing process showed a cytotoxicity grade of Grade 2 after 48 hours. Therefore, it could be seen that the existing process was also suitable for cytocompatibility and toxicity evaluation, and it could be seen that Example 1, which corresponds to the new process, showed higher compatibility, i.e., higher safety, than the existing process.

TABLE 4

| | Cytotoxicity rating | | |
|---|---|---|---|
| Classification | Early stage | 24 hr | 48 hr |
| Existing | 0 | 0 | 2 |
| process | | | |
| Example 1 (New | 0 | 0 | 0 |
| process) | | | |
| Negative | 0 | 0 | 0 |
| control group | | | |
| Positive | 0 | 4 | 4 |
| control group | | | |

### Experimental Example 4: Measurement of Fat Removal Rates According to IPA Washing Conditions

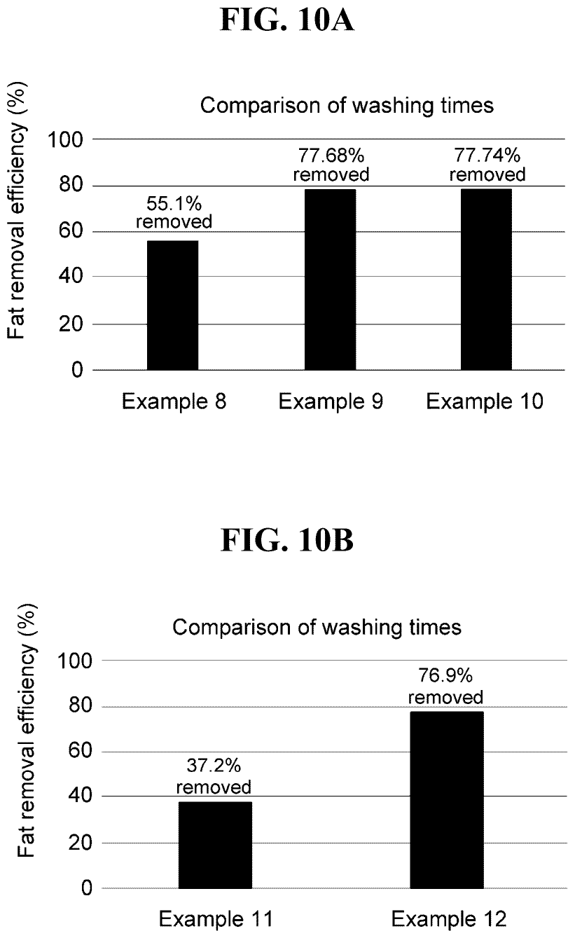

The fat removal conditions were broadly divided into four conditions as shown below, and the evaluation results for each test condition were graphed, and the results are shown in FIGS. 8 to 11.

The samples after completing the second IPA washing were freeze-dried for 24 to 72 h, cut into 0.5×0.5 cm pieces, and measured for the weights of the samples and the weight of the empty container. Then, the sample and the container were installed in the crude fat extraction device, and the crude fat was extracted using the Soxhlet extraction method. After that, the weight of the extracted crude fat was measured and the crude fat content was calculated according to Equation (1) below, and then the fat removal rate according to the IPA-washed group was calculated according to Equation (2) below compared to the crude fat content of the control group sample. That is, in that the fat removal rate increases as the thickness of the tissue becomes thinner, the fat removal rate was calculated as a difference value compared to each control group so that the differences in results due to the differences in the thicknesses of the tissues applied to the Examples could be offset.

$$Crude Fat Content ( \% ) = [ ( Residue Container Weight - Empty Container Weight ) / Sample Weight ] \times 100 \qquad Equation ( 1 )$$

$$Fat Removal Rate ( \% ) = [ ( Control Group Crude Fat Content - Experimental Group Crude Fat Content ) / Control Group Crude Fat Content ] \times 100 \qquad Equation ( 2 )$$

As described below, the applicant of the present disclosure was able to confirm that the fat removal rate was improved by 20% or more compared to the case where only the primary washing with an isotonic solution was performed (control group).

### 4-1. Measurement of Fat Removal Rates According to IPA Washing Concentrations

To check the fat removal rates according to IPA washing concentrations, the fat removal rates of Examples 2 to 4, which were subjected to secondary washing with 50%, 70%, and 100% IPA, were measured, and the results are shown in FIG. 8.

Referring to FIG. 8, the highest fat removal efficiency could be confirmed when washing was performed with 100% IPA.

### 4-2. Measurement of Fat Removal Rates According to Washing Temperatures

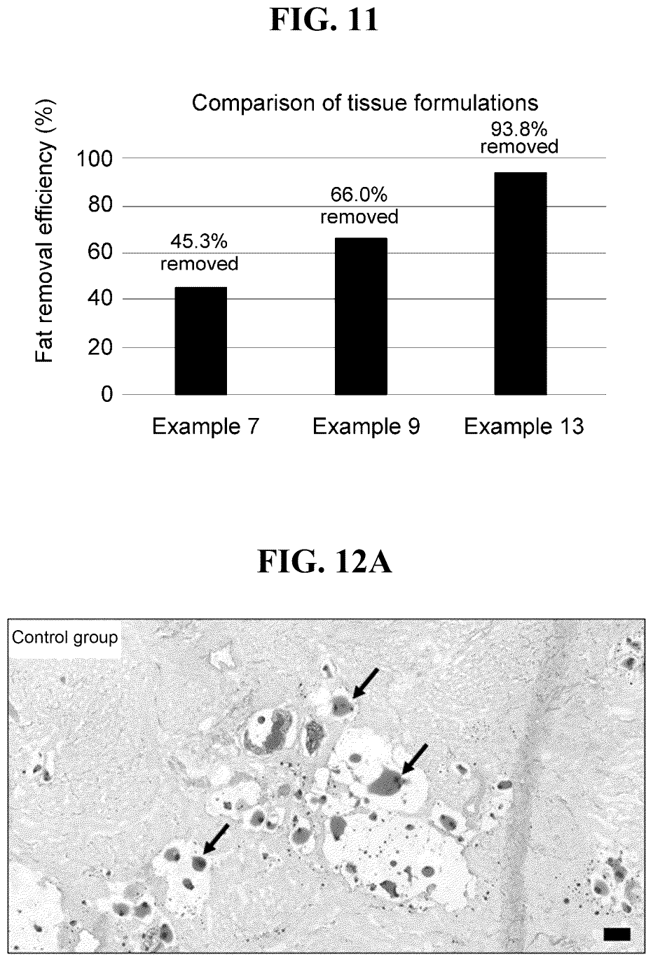

To check the fat removal rates according to washing temperatures, the fat removal rates of Example 5 on which washing was performed at 4° C., Example 6 on which washing was performed at 20 to 25° C., and Example 7 on which washing was performed at 38° C. were measured, and the results are shown in FIG. 9.

Referring to FIG. 9, since the fat removal efficiency is shown to increase as it goes to Example 5, Example 6, and Example 7, it could be seen that the higher the washing temperature, the higher the fat removal efficiency.

Meanwhile, when measuring fat removal rates according to washing temperatures in the present disclosure, if the fat content is measured higher or lower in the control group tissue area within the same lot, the fat removal rates were measured from the results calculated by reflecting the average fat content of the control group tissue considering that an error in which fat removal efficiencies are measured higher or lower across all experimental group conditions may occur.

### 4-3. Measurement of Fat Removal Rates According to Washing Times

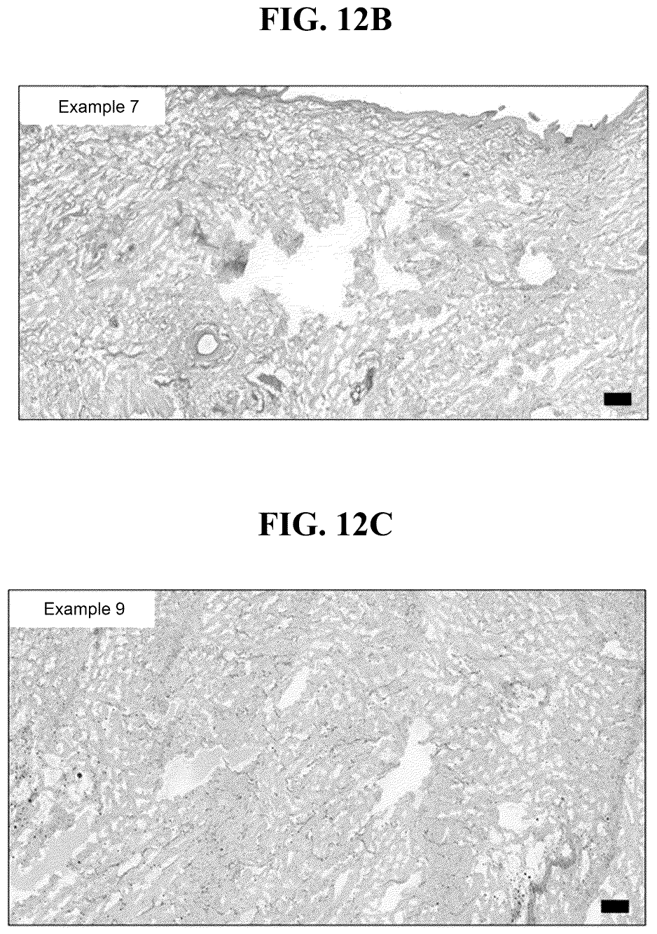

To check the fat removal rates according to washing times, the fat removal rates of the slice formulations, Examples 8 to 10, and the whole formulations, Examples 11 and 12, were measured, and the results are shown in FIGS. 10A and 10B.

Referring to FIGS. 10A and 10B, since the fat removal efficiency in the case of the slice formulation is shown to increase as it goes to Example 8, Example 9, and Example 10, it could be seen that the longer the washing time, the higher the fat removal efficiency. Even in the case of whole formulation similarly to the slice formulation, Example 12 having a longer washing time exhibited a higher fat removal efficiency.

### 4-4. Measurement of Fat Removal Rates According to Tissue Formulations

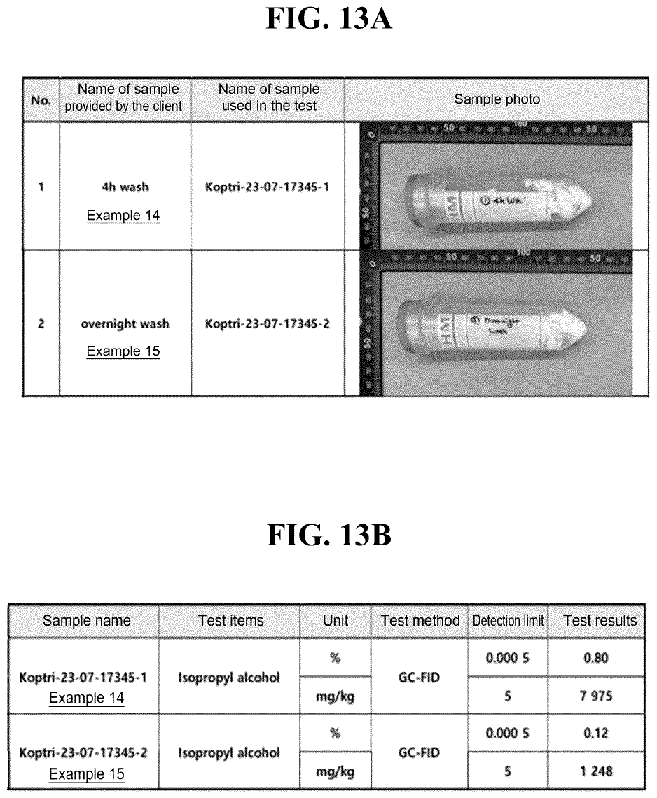

To check the fat removal rates according to tissue formulations, the fat removal rates of the whole formulation, Example 7, the slice formulation, Example 9, and the powder formulation, Example 13, were measured, and the results are shown in FIG. 11.

Referring to FIG. 11, although the fat removal efficiencies were shown to be high as it goes to Example 7, Example 9, and Example 13, it could be seen that the fat removal efficiencies were mostly shown to be excellent in mostly all tissue formulations.

### Experimental Example 5: Measurement of Presence or Absence of Residual Fat Removal According to Tissue Formulations

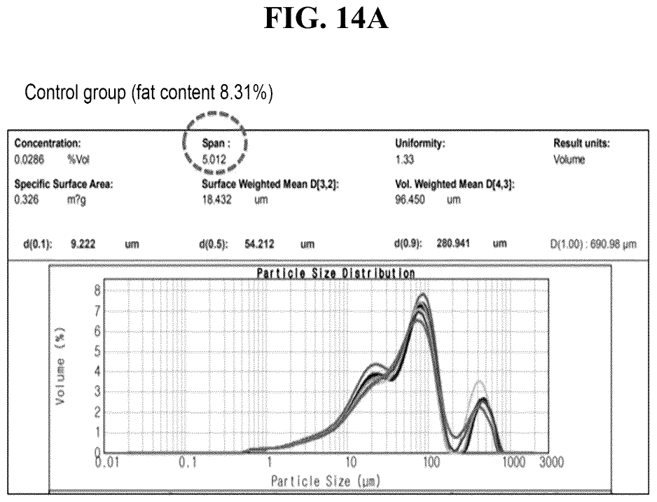

To check the presence or absence of residual fat removal according to tissue formulations, the fats remaining in the tissues of the control group (Example 1), Example 7, and Example 9 were stained and photographed with an optical microscope, and the results are shown in FIGS. 12A to 12C.

Referring to FIGS. 12A to 12C, residual fat was checked in the control group before IPA washing, but residual fats were not checked at all in Examples 7 and 9 after IPA washing. Therefore, it could be seen that most of the residual fats was removed by IPA washing.

### Experimental Example 6: Measurement of IPA Residual Amounts

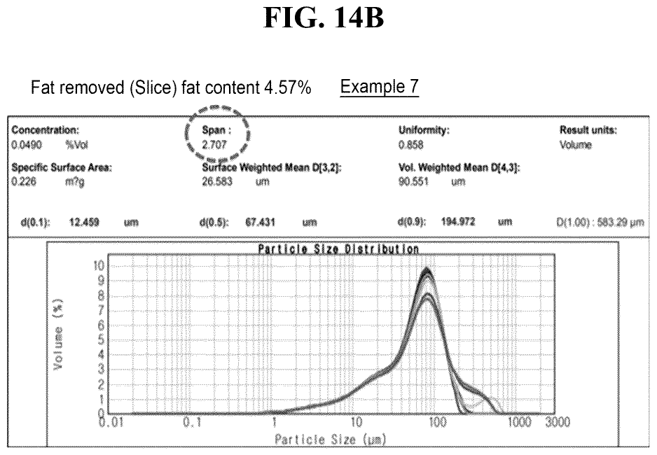

To check the IPA residual amounts according to the DW washing times, as shown in FIG. 13A, the IPA residual amounts were measured for Example 14 on which washing was performed for 4 hours and Example 15 on which washing was performed overnight according to the GC-FID test method, and the results are shown in FIG. 13B.

Referring to FIG. 13B, the IPA residual amount could be confirmed to be 7975 ppm for Example 14 and 1248 ppm for Example 15.

### Experimental Example 7: Evaluation of ADM Powder Particle Distribution Uniformity

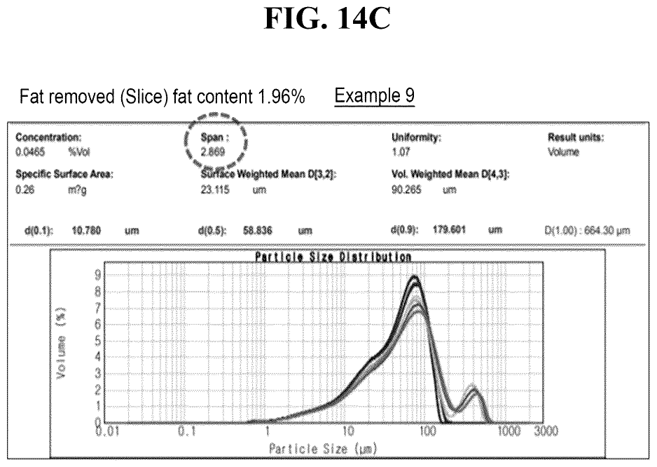

To check the particle distribution uniformity depending on the presence or absence of residual fat removal, the human body-derived acellular dermal matrices prepared according to Examples 1, 7, and 9 were freeze-dried for 24 to 72 hours, then ground to micron units using a freeze grinder, and the particle size and distribution map were measured by applying the Mie theory of the laser diffraction method of a particle size analyzer, and the results are shown in FIGS. 14A to 14C.

The span value is the sample distribution width, and the larger the particle distribution map, the larger the value, and the narrower the distribution map, the closer the value is to 0.

Referring to FIGS. 14A to 14C, although the average particle sizes were measured to be 100 μm or less all for the control group (Example 1), Example 7, and Example 9, showing no significant difference, since the span values were measured to be higher in the control group, it could be confirmed that the particle sizes were relatively uneven. Therefore, it could be found that Examples 7 and 9 are suitable for use in injections such as injectable ADM products prepared using ADM powder.

### Experimental Example 8: Measurement of Amino Acid Content Changes after Secondary Washing

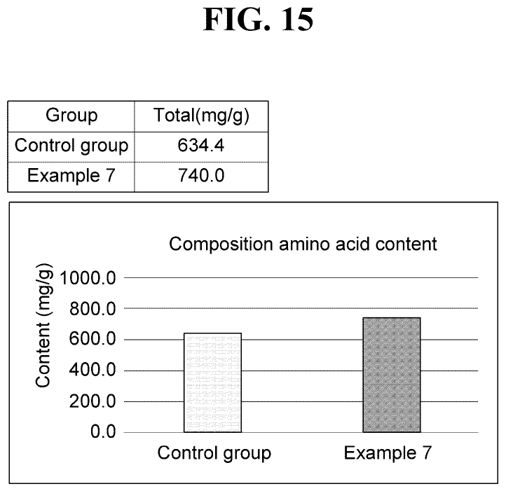

To check the changes in amino acid contents according to before and after IPA, the amino acid contents of the control group (Example 1) and Example 7 were measured using an amino acid auto-analyzer (S433D, Sykam GmbH Co., Germany), and the results are shown in FIG. 15.

Referring to FIG. 15, it could be confirmed that the amino acid content in Example 7 did not decrease compared to the control group, and accordingly it could be seen that the presence or absence of IPA treatment did not affect the amino acids constituting the tissue.

Although exemplary embodiments of the present disclosure have been described in detail above, the scope of rights of the present disclosure is not limited thereto, and various modifications and improved forms made by those skilled in the art utilizing the basic concepts of the present disclosure defined in the following claims also fall within the scope of rights of the present disclosure.

All technical terms used in the present disclosure, unless otherwise defined, are used as the same meaning as commonly understood by those skilled in the art in the relevant fields of the present disclosure. The contents of all publications cited in this specification as references are incorporated in the present disclosure by reference.

1. A method for preparing a human body-derived acellular dermal matrix, comprising steps of:
(i) preparing a human body-derived skin tissue on which separate de-epidermization and delipidation processes have not been performed;
(ii) immersion-treating the skin tissue in a hypotonic solution containing a surfactant;
(iii) primarily washing the skin tissue that has been immersion-treated in the step (ii) with an isotonic solution; and
(iv) secondly washing the skin tissue that has primarily been washed in the step (iii) with a delipidation solution.

2. The method of claim 1, further comprising:
(v) after the step (iv), a step of tertiarily washing the skin tissue secondarily washed in the step (iv) with purified water.

3. The method of claim 1, wherein the surfactant in the step (ii) includes an ionic surfactant, a non-ionic surfactant, or a mixture thereof.

4. The method of claim 1, wherein the hypotonic solution in the step (ii) comprises 5 to 20 mM of Tris-HCl, 0.1 to 1.5% (w/v) of ethylenediaminetetraacetic acid (EDTA), and 0.01 to 0.05 M of sodium hydroxide (NaOH).

5. The method of claim 1, wherein the concentration of the surfactant in the step (ii) is 0.1 to 0.5% (w/v) in the hypotonic solution.

6. The method of claim 1, wherein the isotonic solution in the step (iii) comprises 25 to 100 mM of Tris-HCl, 0.1 to 1.5% (w/v) of ethylenediaminetetraacetic acid (EDTA), 0.01 to 0.05 M of sodium hydroxide (NaOH), and 0.05 to 0.3 M of sodium chloride (NaCl).

7. The method of claim 1, wherein the secondary washing in the step (iv) is performed at 4 to 40° C. for 2 to 24 hours using a delipidation solution having a concentration of 50 to 100%.

8. The method of claim 1, wherein the delipidation solution in the step (iv) comprises a polar solvent.

9. The method of claim 2, wherein the tertiary washing in the step (v) is performed for 4 to 24 hours.

10. The method of claim 1, wherein the step (iv) is omitted, and the fat removal rates measured by the following Equations (1) and (2) are improved by 20% or more compared to a control group that is the human body-derived acellular dermal matrix prepared by the steps (i), (ii), and (iii):
$$Crude Fat Content ( \% ) = [ ( Residue Container Weight - Empty Container Weight ) / Sample Weight ] \times 100 \qquad Equation ( 1 )$$
$$Fat Removal Rate ( \% ) = [ ( Control Group Crude Fat Content - Experimental Group Crude Fat Content ) / Control Group Crude Fat Content ] \times 100 \qquad Equation ( 2 )$$

11. A human body-derived acellular dermal matrix prepared by washing multiple times a human body-derived skin tissue on which separate de-epidermation and delipidation processes have not been performed, wherein the washing includes washing with an isotonic solution and washing with a delipidation solution, and the delipidation solution comprises a polar solvent.

12. The human body-derived acellular dermal matrix of claim 11, wherein the residual amount of SDS detergent measured as absorbance at 650 nm for an eluate eluted with an MEM culture medium is 40 mg/L or less.

13. The human body-derived acellular dermal matrix of claim 11, having a cell viability of 90% or more measured as absorbance at 540 nm for the eluate eluted with the MEM culture medium.
